(19) United States
(12) Patent Application Publication
(10) Pub. No.: US 2014/0196740 A1
(43) Pub. Date: Jul. 17, 2014

(54) HAIR CARE PRODUCTS WITH ENHANCED CARE PERFORMANCE

(71) Applicant: Henkel AG & Co. KGaA, Duesseldorf (DE)

(72) Inventors: Manuela Mette, Kleinfeld (DE); Thomas Hippe, Appen (DE)

(73) Assignee: Henkel AG & Co. KGaA, Duesseldorf (DE)

(21) Appl. No.: 14/214,091

(22) Filed: Mar. 14, 2014

Related U.S. Application Data
(63) Continuation of application No. PCT/EP2012/066730, filed on Aug. 29, 2012.

(30) Foreign Application Priority Data
Sep. 16, 2011 (DE) 10 2011 082 898.2

(51) Int. Cl. A61K 8/898; A61Q 5/12; A61K 8/37; A61K 8/67; A61K 8/58; A61K 8/84; A61K 8/41

(52) U.S. Cl. CPC A61K 8/898; A61K 8/84; A61Q 5/12; A61K 8/416; A61K 8/673; A61K 8/585; A61K 8/37
USPC 132/202; 424/70.17; 424/70.122

(57) ABSTRACT

The present invention describes compositions for the treatment of keratinic fibers, in particular human hair, that significantly improve the conditioning properties and also the shine and feel of keratinic fibers, in particular human hair, as compared with prior-art compositions. The compositions according to the invention include as essential ingredients cationic ammonium compounds, amidoamines, ester oils, cationic polymers and sugar surfactants.

### FIELD OF THE INVENTION

The present invention generally relates to compositions for the treatment of keratinic fibers, in particular human hair, that significantly improve the conditioning properties and also the shine and feel of keratinic fibers, in particular human hair, as compared with prior-art compositions. The compositions according to the invention include as essential ingredients cationic ammonium compounds, amidoamines, ester oils, cationic polymers and sugar surfactants.

### BACKGROUND OF THE INVENTION

The cosmetic treatment of keratinic fibers, in particular human hair, is an important element of human body care. Thus human hair is now treated in many different ways with hair cosmetics preparations.

There is still a demand for active ingredients or for combinations of active ingredients for cosmetic agents having good care properties and good biodegradability. The acceptability of the cosmetic compositions is moreover an exceptionally important criterion.

Compositions for use on keratinic fibers, in particular human hair, must not only have a good cleaning, conditioning and care ability but must moreover be well tolerated and not lead to severe degreasing or dryness and split ends, even in frequent use. The feel of the keratinic fibers after cosmetic treatments is an essential criterion in whether the corresponding composition is perceived as pleasing by the consumer. The sensory quality and in particular the feel of a composition are thus essential effects that the consumer can experience. The aim is therefore to find precisely those compositions which not only care for keratinic fibers, in particular human hair, but moreover through their sensory and in particular tactile qualities bring about a tangible and perceptible change in the surface of keratinic fibers, in particular human hair. In particular, the compositions should moreover regenerate and balance out the structure inside the keratinic fibers, in particular human hair. At the same time the compositions should be simple and inexpensive to produce.

Quite unexpectedly, however, it has now been found that a cosmetic composition that includes, in a cosmetic carrier,
a) 0.1 to 6.0 wt. % of at least one cationic surfactant selected from behentrimonium chloride and/or dimethyl dibehenyl ammonium chloride and/or tribehenyl methyl trimonium chloride and/or cetyl trimethylammonium chloride and/or dicetyl dimethylammonium chloride and/or tricetyl methylammonium chloride and/or trimethyl stearyl ammonium chloride, dimethyl distearyl ammonium chloride and/or tristearyl methylammonium chloride and mixtures thereof,
b) 0.01 to 6.0 wt. % of at least one amidoamine and/or one permanently cationic amidoamine,
c) 0.01 to 7.5 wt. % of at least one ester oil,
d) 0.01 to 5.0 wt. % of at least one cationic polymer selected from Polyquaternium-67 and/or Polyquaternium-72 and/or Polyquaternium-74 and/or Polyquaternium-37, and
e) 0.001 to 3.0 wt. % of at least one sugar surfactant,
   all stated amounts relating to the total weight of the cosmetic composition, is ideally suitable.

The use of this combination leads to surprisingly good properties in the treated hair, in particular to improved combability, to improved shine and to an improved elasticity as well as to a marked increase in the washing resistance of colored hair and to a longer hold combined at the same time with a better shaping performance in waving processes such as finger waving and permanent waving.

Furthermore, other desirable features and characteristics of the present invention will become apparent from the subsequent detailed description of the invention and the appended claims, taken in conjunction with the accompanying drawings and this

### BACKGROUND OF THE INVENTION

### Brief Summary of the Invention

A cosmetic composition for conditioning keratinic fibers, including in a cosmetic carrier: a) 0.1 to 6.0 wt. % of at least one cationic surfactant selected from behentrimonium chloride and/or dimethyl dibehenyl ammonium chloride and/or tribehenyl methyl trimonium chloride and/or cetyl trimethylammonium chloride and/or dicetyl dimethylammonium chloride and/or tricetyl methylammonium chloride and/or trimethyl stearyl ammonium chloride, dimethyl distearyl ammonium chloride and/or tristearyl methylammonium chloride and mixtures thereof; b) 0.01 to 6.0 wt. % of at least one amidoamine and/or one permanently cationic amidoamine; c) 0.01 to 7.5 wt. % of at least one ester oil; d) 0.01 to 5.0 wt. % of at least one cationic polymer selected from Polyquaternium-67 and/or Polyquaternium-72 and/or Polyquaternium-74 and/or Polyquaternium-37; and e) 0.001 to 3.0 wt. % of at least one sugar surfactant, all stated amounts relating to the total weight of the cosmetic composition.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

Hair treatment agents within the meaning of the present invention are for example hair shampoos, hair conditioners, conditioning shampoos, hair rinses, hair masks, hair packs, hair tonics, hair coloring shampoos or combinations thereof. In particular, the hair treatment agents according to the invention are understood to be hair conditioning compositions such as hair rinses, hair masks, hair packs, hair oils and lotions, both as leave-on products, i.e. products that remain on the hair until the next time it is washed, and as rinse-off products, i.e. products that are rinsed out again a few seconds up to a few hours after application.

According to the invention combability is understood to mean both the combability of wet fibers and the combability of dry fibers.

Feel is defined as the tactility of a group of fibers, the person skilled in the art feeling and assessing the parameters fullness and softness of the group of fibers by sensory means.

Shaping is understood to be the ability to change the shape of a group of previously treated keratin-including fibers, in particular human hair. In hair cosmetics this is also referred to as styleability.

Restructuring within the meaning of the invention is understood to be a reduction in the damage that is caused to keratinic fibers by a very wide variety of influences. The restoration of natural strength, for example, plays an important part in this. Restructured fibers are characterized by an improved shine, an improved feel and easier combability. They also have improved strength and elasticity. Furthermore, a successful restructuring can be physically detected as an increase in the melting point in comparison to damaged fibers. The higher the melting point of the hair, the stronger the structure of the fiber.

Wash fastness within the meaning of the invention is understood to be the retention of the shade and/or intensity of the original color when the keratinic fiber is exposed to the repeated influence of aqueous agents, in particular surfactant-including agents such as shampoos.

The compositions according to the invention including the active ingredient complex according to the invention are characterized moreover by a markedly improved condition of the keratinic fibers in terms of the moisture balance of the keratinic fibers. Furthermore, the active ingredient complex according to the invention leads to a marked protection of the keratinic fibers against the effects of heat, for example when blow-drying keratinic fibers. Protecting the surface of keratinic fibers against the effects of heat is very important, particularly when using hair straighteners or hair dryers. Finally, it was established that the compositions according to the invention surprisingly lead to a marked lengthening of the time taken for the keratinic fibers to become dirty again.

An aqueous cosmetic carrier includes at least 50 wt % of water.

Within the meaning of the present invention aqueous-alcoholic cosmetic carriers are understood to be aqueous solutions including 3 to 70 wt. % of a C₁-C₆ alcohol, in particular methanol, ethanol or propanol, isopropanol, butanol, isobutanol, tert-butanol, n-pentanol, isopentanols, n-hexanol, isohexanols, glycol, glycerol, 1,2-pentanediol, 1,5-pentanediol, 1,2-hexanediol or 1,6-hexanediol. The agents according to the invention can additionally include further organic solvents, such as for example methoxybutanol, benzyl alcohol, ethyl diglycol or 1,2-propylene glycol. All water-soluble organic solvents are preferred here. Water is more preferred.

Cationic surfactants of formula (Tkat1) are the first group of required ingredients of the compositions according to the invention.

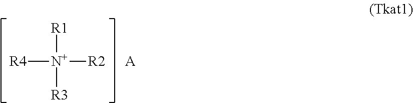

In the formula (Tkat1) the residues R1, R2, R3 and R4 generally each independently of one another denote hydrogen, a methyl group, a saturated, branched or unbranched alkyl residue with a chain length of 8 to 30 carbon atoms, which can optionally be substituted with one or more hydroxyl groups, and A denotes a physiologically acceptable anion, for example halides such as chloride or bromide and methosulfates.

Compounds according to the invention are behentrimonium chloride and/or dimethyl dibehenyl ammonium chloride and/or tribehenyl methyl trimonium chloride and/or cocotrimonium chloride and/or dicocodimonium chloride and/or tricocomonium chloride and/or cetyl trimethylammonium chloride and/or dicetyl dimethylammonium chloride and/or tricetyl methylammonium chloride and/or trimethyl stearyl ammonium chloride, dimethyl distearyl ammonium chloride and/or stearyl trimethylammonium chloride and/or mixtures thereof. Preferred compounds are behentrimonium chloride and/or dimethyl dibehenyl ammonium chloride and/or cetrimonium chloride and/or dicetyl dimethylammonium chloride and/or stearyl trimethylammonium chloride and/or dimethyl distearyl ammonium chloride and/or mixtures thereof. Behentrimonium chloride and/or cetyl trimethylammonium chloride and/or stearyl trimethylammonium chloride are more preferred. Behentrimonium chloride and/or cetyl trimonium chloride and the mixture thereof are highly preferred.

The cationic surfactants according to the invention are included in the compositions according to the invention in an amount from 0.1 to 6.0 wt. %, relative to the total weight of the cosmetic preparations. Amounts from 0.2 to 4.5 wt. % are preferred. Amounts from 0.2 to 3.0 wt. % are more preferred, with amounts from 0.3 to 2.5 wt. % being highly preferred, relative in each case to the total weight of the cosmetic preparation.

The second group of essential ingredients of the present invention are amidoamines and/or permanently cationized amidoamines, in particular with the following structural formulae:

R¹—NH—(CH₂)ₙ—N⁺R²R³R⁴A (Tkat3)

in which R1 denotes an acyl or alkyl residue which in each case has 6 to 30 C atoms and which in each case can be branched or unbranched, saturated or unsaturated, and wherein the acyl residue and/or alkyl residue can include at least one OH group, and R2, R3 and R4 in each case independently of one another denote
1) hydrogen or
2) an alkyl residue having 1 to 4 C atoms, which can be the same or different, saturated or unsaturated, and
3) a branched or unbranched hydroxyalkyl group having 1 to 4 carbon atoms with at least one and at most three hydroxyl groups, for example —CH₂OH, —CH₂CH₂OH, —CHOHCHOH, —CH₂CHOHCH₃, —CH(CH₂OH)₂, —COH(CH₂OH)₂, —CH₂CHOHCH₂OH, —CH₂CH₂CH₂OH and hydroxybutyl residues, and
A denotes a physiologically acceptable anion and
n denotes an integer between 1 and 10.

Suitable physiologically acceptable counterions A are for example halide ions, sulfate ions, phosphate ions, methosulfate ions as well as organic ions such as lactate, citrate, tartrate and acetate ions. Methosulfates and halide ions, in particular chloride, are preferred.

A composition in which the amidoamine and/or the quaternized amidoamine according to the general formula (Tkat3) is an amidoamine and/or a quaternized amidoamine in which R1 denotes a branched or unbranched, saturated or unsaturated acyl residue having 6 to 30 C atoms, which can include at least one OH group, is preferred.

A fatty acid residue from oils and waxes, in particular from natural oils and waxes, is preferred here. Suitable examples include lanolin, beeswax or candelilla wax.

Also preferred are amidoamines and/or quaternized amidoamines in which R2, R3 and/or R4 in formula (Tkat3) denote a residue according to the general formula CH₂CH₂OR5, in which R5 can have the meaning of alkyl residues having 1 to 4 carbon atoms, hydroxyethyl or hydrogen. The preferred value of n in the general formula (Tkat3) is an integer between 2 and 5.

The alkyl amidoamines can be present as is and can also be converted into a quaternary compound in the composition by protonation in a correspondingly acid solution. Cationic acyl amidoamines are preferred according to the invention.

Examples of amidoamines according to the invention are: Lauramidopropyl Dimethylamine (Mackine 801), Lauramidopropyl Dimethylamine Propionate, Stearamidopropyl Dimethylamine (Adogen® S18V or Tego® Amid S 18 or Incromine® SB), Myristamidopropyl Dimethylamine (Schercodine M), Stearamidoethyl Diethylamine (Lexamine 22), Stearamidoethyl Diethylamine Phosphate, Cocamidopropyl Dimethylamine (Mackine® 101), Ricinolamidopropyl Dimethylamine (Mackine® 201), Isostearamidopropyl Dimethylamine (Mackine 401), Oleamidopropyl Dimethylamine (Mackine® 501), Behenamidopropyl Dimethylamine (Mackine 601, Incromine® BD), Cocamidopropyl Dimethylamine Propionate (Mackalene® 117), Cocamidopropyl Dimethylamine Lactate (Mackalene® 116), Ricinoleamidopropyl Dimethylamine Lactate (Mackalene 216), Stearamidopropyl Dimethylamine Lactate (Mackalene 316), Behenamidopropyl Dimethylamine Lactate (Mackalene® 616), Sunflowerseedamidopropyl Dimethylamine Lactate (Mackalene 1216), Palmamidopropyl Dimethylamine, Palmamidopropyl Dimethylamine Lactate, Palmamidopropyl Dimethylamine Propionate, Oleamidopropyl Dimethylamine Glycolate, Oleamidopropyl Dimethylamine Lactate, and examples of permanently cationic amidoamines are: Quaternium-33 (Swanol® Lanoquat DES-50), Behenamidopropyl Ethyldimonium Ethosulfate (Schercoquat® BAS), Behenamidopropyl PG-Dimonium Chloride (Lexquat® AMG-BEO), Oleamidopropyl Ethyldimonium Ethosulfate, Oleamidopropyl PG-Dimonium Chloride (Lexquat® AMG-O), Cocamidopropyl Ethyldimonium Ethosulfate (Schercoquat® CAS), Cocamidopropyltrimoniumchloride (Empigen® CSC), Ricinoleamidopropylethyl-dimonium Ethosulfate, Rinoleamidopropyltrimoniumchloride, Ricinoleamido-propyltrimoniummethosulfate (Rewoquat® RTM 50), Stearamidopropyl Ethyldimonium Ethosulfate (Schercoquat® SAS), Stearamidopropyl Trimonium Methosulfate (Catagene® SA-70) or Undecyleneamidopropyltrimonium Methosulfate (Rewoquat® UTM 50), Lauramidopropyl PG-Dimonium Chloride, Canolamidopropyl Ethyldimonium Ethosulfate (Schercoquat® COAS).

Preferred amidoamines are Lauramidopropyl Dimethylamine, Myristamidopropyl Dimethylamine, Stearamidopropyl Dimethylamine, Cocamidopropyl Dimethylamine, Ricinolamidopropyl Dimethylamine, Isostearamidopropyl Dimethylamine, Oleamidopropyl Dimethylamine, Behenamidopropyl Dimethylamine, Palmamidopropyl Dimethylamine, Quaternium-33, Behenamidopropyl Ethyldimonium Ethosulfate, Oleamidopropyl Ethyldimonium Ethosulfate, Cocamidopropyltrimoniumchloride, Rinoleamidopropyltrimoniumchloride, Stearamidopropyl Trimonium Methosulfate.

The amidoamines Stearamidopropyl Dimethylamine, Cocamidopropyl Dimethylamine, Ricinolamidopropyl Dimethylamine, Isostearamidopropyl Dimethylamine, Oleamidopropyl Dimethylamine, Behenamidopropyl Dimethylamine, Palmamidopropyl Dimethylamine, Quaternium-33 and Behenamidopropyl Ethyldimonium Ethosulfate are more preferred.

Stearamidopropyl Dimethylamine, Cocamidopropyl Dimethylamine, Isostearamidopropyl Dimethylamine, Behenamidopropyl Dimethylamine and Behenamidopropyl Ethyldimonium Ethosulfate are highly preferred.

Stearamidopropyl Dimethylamine is most preferred.

The aforementioned amidoamines can be used individually or in any combinations with one another, wherein they are included in amounts of between 0.01 and 6.0 wt. %, preferably in amounts from 0.01 to 3.0 wt. % and particularly preferably in amounts from 0.1 to 2.5 wt. %. The very best results are obtained with amounts from 0.2 to 1.5 wt. %, relative in each case to the total composition of the individual agent.

The third group of ingredients that are absolutely necessary according to the invention are ester oils. Ester oils are understood to be the esters of C₆-C₃₀ fatty acids with C₂-C₃₀ fatty alcohols. The monoesters of fatty acids with alcohols having 2 to 24 C atoms are preferred. Examples of fatty acid components used in the esters are hexanoic acid, octanoic acid, 2-ethylhexanoic acid, decanoic acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, eicosanoic acid, gadoleic acid, docosanoic acid and erucic acid and technical mixtures thereof. Examples of the fatty alcohol components in the ester oils are isopropyl alcohol, caproic alcohol, caprylic alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol and technical mixtures thereof. More preferred according to the invention are isopropyl myristate (Rilanit® IPM), isononanoic acid C₁₆₋₁₈ alkyl ester (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V).

The ester oils can naturally also be alkoxylated with ethylene oxide, propylene oxide or mixtures of ethylene oxide and propylene oxide. The alkoxylation can take place both on the fatty alcohol part and on the fatty acid part and also on both parts of the ester oils. It is preferred according to the invention, however, for the fatty alcohol to be alkoxylated first and then esterified with fatty acid. These compounds are shown in general in formula (D4-II).

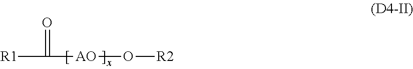

R1 here denotes a saturated or unsaturated, branched or unbranched, cyclic saturated, cyclic unsaturated acyl residue having 6 to 30 carbon atoms,
AO denotes ethylene oxide, propylene oxide or butylene oxide,
X denotes a number between 1 and 200, preferably between 1 and 100, more preferably between 1 and 50, particularly preferably between 1 and 20, highly preferably between 1 and 10 and most preferably between 1 and 5,
R2 denotes a saturated or unsaturated, branched or unbranched, cyclic saturated, cyclic unsaturated alkyl, alkenyl, alkynyl, phenyl or benzyl residue having 6 to 30 carbon atoms. Examples of fatty acid components used as residue R1 in the esters are hexanoic acid, octanoic acid, 2-ethylhexanoic acid, decanoic acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, eicosanoic acid, gadoleic acid, docosanoic acid and erucic acid and technical mixtures thereof. Examples of the fatty alcohol components as residue R2 in the ester oils are benzyl alcohol, isopropyl alcohol, caproic alcohol, caprylic alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol and technical mixtures thereof. An ester oil that is more preferred according to the invention is available for example under the INCI name PPG-3 Benzyl Ether Myristate.

Ester oils are also understood to include:
Dicarboxylic acid esters such as di-n-butyl adipate, di-(2-ethylhexyl) adipate, di-(2-ethylhexyl) succinate and diisotridecyl acelate and also diol esters such as ethylene glycol dioleate, ethylene glycol diisotridecanoate, propylene glycol di-(2-ethylhexanoate), propylene glycol diisostearate, propylene glycol dipelargonate, butanediol diisostearate, neopentyl glycol dicaprylate, and
Symmetrical, asymmetrical or cyclic esters of carbonic acid with fatty alcohols, for example glycerol carbonate or dicaprylyl carbonate (Cetiol® CC),
Fatty acid partial glycerides, namely monoglycerides, diglycerides and technical mixtures thereof. Typical examples are mono- and/or diglycerides based on hexanoic acid, octanoic acid, 2-ethylhexanoic acid, decanoic acid, lauric acid, isotridecanoic acid, myristic acid, palmitic acid, palmoleic acid, stearic acid, isostearic acid, oleic acid, elaidic acid, petroselic acid, linoleic acid, linolenic acid, elaeostearic acid, eicosanoic acid, gadoleic acid, docosanoic acid and erucic acid and technical mixtures thereof. Oleic acid monoglycerides are preferably used.

Naturally it is also possible according to the invention to use a plurality of ester oils at the same time. Preferred ester oils are isopropyl myristate, glycerol carbonate, dicaprylyl carbonate, isopropyl palmitate, PPG-3 benzyl ether myristate, cetyl oleate and oleyl erucate and mixtures of at least two of these ester oils. Mixtures of ester oils in which one of the ester oils is dicaprylyl carbonate or isopropyl myristate are most preferred. Mixtures of and with these two ester oils are highly preferred. It is most preferable to use a mixture of ester oils with the last two specified oils if the isopropyl myristate and the caprylyl carbonate are in a ratio of 10:1 to 1:1, preferably 5:1 to 1:1, more preferably 5:1 to 3:1, relative to the amounts of ester oils in each case.

The ester oils are used in the agents according to the invention in an amount from 0.01 to 7.5 wt. %, preferably 0.01 to 5.0 wt. %, more preferably 0.05 to 5.0 wt. %, highly preferably from 0.2 to 5.0 wt. %.

Cationic polymers are used as the fourth required component of the compositions according to the invention. The cationic polymers are selected from the following cationic polymers:

Suitable cationic polymers that are derived from natural polymers are cationic derivatives of polysaccharides, for example cationic derivatives of cellulose, starch or guar. Cationic polysaccharides have the general formula

G-O—B—N⁺RₐR_{b}R_{c}A⁻

G is an anhydroglucose residue, for example starch or cellulose anhydroglucose;
B is a divalent group of compounds, for example alkylene, oxyalkylene, polyoxyalkylene or hydroxyalkylene;
Rₐ, R_{b} and R_{c} are independently of one another alkyl, aryl, alkylaryl, arylalkyl, alkoxyalkyl or alkoxyaryl, each having up to 18 C atoms, the total number of C atoms in Rₐ, R_{b} and R_{c} preferably being a maximum of 20;
A⁻ is a conventional counter anion and is preferably chloride.

Of the polymers derived from the natural cationic polymers, cationic celluloses are preferred. Cationic celluloses are available on the market with varying degrees of substitution, cationic charge density, nitrogen content and molecular weights. In particular, the following cationic celluloses are highly preferred according to the invention.

Polyquaternium-67 is commercially available for example under the names Polymer® SL or Polymer® SK (Amerchol). A further highly preferred cellulose is available under the trade name Mirustyle® CP from Croda. This is a trimonium and cocodimonium hydroxyethylcellulose as a derivatized cellulose with the INCI name Polyquaternium-72. Polyquaternium-72 can be used both in solid form and pre-dissolved in aqueous solution.

Suitable cationic polymers derived from synthetic polymers are for example copolymers of

A1) 0.1 to 50%, preferably 10 to 50% (relative to the total number of monomers in the copolymer) of monomers of formula (Ia)

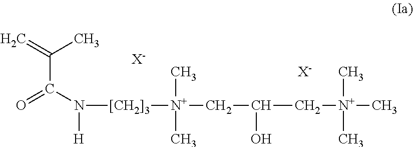

in which
X denotes chloride, sulfate, methosulfate,
A2) monomers from the group comprising acrylic acid, methacrylic acid and the alkali metal salts and ammonium salts of these acids,
wherein monomer A2 makes up 50 to 99.9%, preferably 50 to 90% (relative to the total number of monomers in the copolymer) of the copolymer.

Irrespective of which of the preferred copolymers A of formula (Ia) are used, hair treatment agents according to the invention are preferred which are characterized in that the ratio of (y:z) is 4:1 to 1:2, preferably 4:1 to 1:1.

Irrespective of which copolymers A are used in the agents according to the invention, hair treatment agents according to the invention are preferred in which copolymer A has a molar mass from 10,000 to 20 million gmol⁻¹, preferably from 100,000 to 10 million gmol⁻¹, more preferably from 500,000 to 5 million gmol⁻¹ and in particular from 1.1 million to 2.2 million once.

A highly preferred polymer, which is synthesized as described above, is commercially available under the name Polyquaternium-74.

A further highly preferred cationic synthetic polymer is an optionally crosslinked homopolymer, poly(methacryloyloxyethyl trimethylammonium chloride) with the INCI name Polyquaternium-37. Such products are commercially available for example under the names Rheocare® CTH (Cosmetic Rheologies) and Synthalen® CR (3V Sigma).

The homopolymer is preferably used in the form of a non-aqueous polymer dispersion. Such polymer dispersions are commercially available under the names Salcare® SC 95 and Salcare® SC 96.

Hair treatment agents according to the invention include Polyquaternium-37 in amounts, relative to their weight, of 0.001 to 5 wt. %, preferably 0.0025 to 2.5 wt. %, more preferably 0.005 to 1 wt. %, more preferably 0.0075 to 0.75 wt. % and in particular 0.01 to 0.5 wt. %.

The final required component of the composition according to the invention are sugar surfactants. Non-ionic surfactants based on sugars are highly preferably used as non-ionic surfactants. These are firstly preferably an alkyl or alkenyl oligoglycoside. These non-ionic emulsifiers are known non-ionic surfactants according to formula (I),

R¹O-[G]ₚ (1)

in which R¹ denotes an alkyl or alkenyl residue having 4 to 22 carbon atoms, G denotes a sugar residue having 5 or 6 carbon atoms and p denotes numbers from 1 to 10. The alkyl and alkenyl oligoglycosides can derive from aldoses or ketoses having 5 or 6 carbon atoms, preferably from glucose. The preferred alkyl and/or alkenyl oligoglycosides are thus alkyl and/or alkenyl oligoglucosides. The index value p in the general formula (I) indicates the degree of oligomerization (DP), i.e. the distribution of mono- and oligoglycosides, and denotes a number between 1 and 10. While p in the individual molecule must always be an integer number and above all can assume the values p=1 to 6 here, the value p for a particular alkyl oligoglycoside is a calculated quantity determined by analysis, which in most cases is a fraction. Alkyl and/or alkenyl oligoglycosides having an average degree of oligomerization p of 1.1 to 3.0 are preferably used. Alkyl and/or alkenyl oligoglycosides having a degree of oligomerization below 1.7 are preferred, and in particular between 1.2 and 1.4. The alkyl or alkenyl residue R¹ can derive from primary alcohols having 4 to 22, preferably 8 to 22 carbon atoms. Typical examples are butanol, caproic alcohol, caprylic alcohol, caprinic alcohol, undecyl alcohol, lauryl alcohol, myristyl alcohol, cetyl alcohol, palmoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol, natural fatty alcohols such as coconut alcohol and technical mixtures thereof. Examples of commercially available products are the Oramix° types from Seppic, for example Oramix® NS 10, the Plantacare® types, for example Plantacare® 2000UP, Plantacare® 1200UP, Plantacare® 810UP, Plantacare® 818UP.

The sugar-based emulsifier can moreover be a fatty acid-N-alkylpolyhydroxyalkylamide of formula (II),

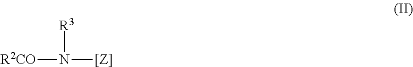

in which R²CO denotes an aliphatic acyl residue having 6 to 22 carbon atoms, R³ denotes hydrogen, an alkyl or hydroxyalkyl residue having 1 to 4 carbon atoms and [Z] denotes a linear or branched polyhydroxyalkyl residue having 3 to 12 carbon atoms and 3 to 10 hydroxyl groups.

The fatty acid-N-alkylpolyhydroxyalkylamides preferably derive from reducing sugars having 5 or 6 carbon atoms, in particular from glucose. The preferred fatty acid-N-alkylpolyhydroxyalkylamides are therefore fatty acid-N-alkyl glucamides as described by formula (III):

R²CO—NR³—CH₂—(CHOH)₄CH₂OH (III)

Glucamides of formula (III) in which R³ denotes hydrogen or an alkyl group and R²CO denotes an acyl residue of hexanoic acid, octanoic acid, decanoic acid, lauric acid, myristic acid, palmitic acid, palmoleic acid, stearic acid, isostearic acid, oleic acid, elaidic acid, petroselic acid, linoleic acid, linolenic acid, eicosanoic acid, gadoleic acid, docosanoic acid or erucic acid and technical mixtures thereof are preferably used as fatty acid-N-alkylpolyhydroxyalkylamides. The polyhydroxyalkylamides can also derive from maltose and palatinose.

The sugar-based non-ionic surfactant is preferably included in the agents used according to the invention in amounts from 0.001 to 3.0 wt. %, relative to the total agent. Amounts from 0.01 to 2.0 wt. % are more preferred.

In addition to the essential ingredients described above, the compositions according to the invention can of course optionally also include further conventional ingredients. These are added in particular to impart further desired properties to the compositions, such as an anti-dandruff effect or to increase volume, etc. These ingredients are described below.

Saturated, mono- or polyunsaturated, branched or unbranched fatty alcohols can be used as fatty alcohols (Fatal). Saturated and unbranched fatty alcohols are preferably used that have a carbon chain length of C₆-C₁₈, preferably C₃-C₁₈ and particularly preferably C₁₀-C₁₆. Mono- or polyunsaturated fatty alcohols and branched and unsaturated or branched and saturated fatty alcohols are preferably used that have a carbon chain length of C₆-C₃₀, preferably C₁₀-C₂₂ and particularly preferably C₁₂-C₂₂. Suitable for use within the meaning of the invention are for example decanol, octanol, octenol, dodecenol, decenol, octadienol, dodecadienol, decadienol, oleyl alcohol, erucic alcohol, ricinol alcohol, lauryl alcohol, myristyl alcohol, caprylic alcohol, capric alcohol, linoleyl alcohol and linolenyl alcohol, wherein this list is intended to be of an exemplary and non-limiting nature. Likewise suitable for use according to the invention are fatty alcohol cuts constituting a mixture of different fatty alcohols. Such substances are available commercially for example under the names Stenol® or Lanette® or Nafol® or Lorol®, e.g. Lorol® C8, Lorol® C14, Lorol® C8-18, HD-Ocenol®, Crodacol®, Novol®, Eutanol® G, Guerbitol® 16, Guerbitol® 18, Guerbitol® 20, Isofol® 12, Isofol® 16, Isofol® 24, Isofol® 36, Isocarb® 12, Isocarb® 16 or Isocarb® 24. Wool wax alcohols can of course also be used according to the invention, such as are available commercially for example under the names Corona®, White Swan®, Coronet® or Fluilan®. The softening point of the fatty alcohols for use according to the invention is at most 45° C., preferably 15 to 40° C., more preferably 15 to 35° C. and highly preferably 15 to 28° C.

The fatty alcohols are used in amounts from 0.1 to 30 wt. %, relative to the total preparation, preferably in amounts from 0.1 to 20 wt. %.

The amount of fatty alcohols used here is 0.1 to 15 wt. %, relative to the total agent. The amount is preferably 0.1 to 10 wt. %, wherein amounts of 0.1 to 5 wt. % can be particularly advantageous.

Linear and/or branched, saturated and/or unsaturated fatty acids having 6 to 30 carbon atoms can be used as fatty acids (Fatac). Fatty acids having 10 to 22 carbon atoms are preferred. Examples which can be cited include the isostearic acids, such as the commercial products Emersol® 871 and Emersol® 875, and isopalmitic acids such as the commercial product Edenor® IP 95, as well as all other fatty acids sold under the Edenor® trade names (Cognis). Further typical examples of such fatty acids are hexanoic acid, octanoic acid, 2-ethylhexanoic acid, decanoic acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, eicosanoic acid, gadoleic acid, docosanoic acid and erucic acid and technical mixtures thereof. The fatty acid cuts obtainable from coconut oil or palm oil are conventionally more preferred; as a rule the use of stearic acid is preferred in particular.

The amount used here is 0.1 to 15 wt. %, relative to the total agent. The amount is preferably 0.5 to 10 wt. %, wherein amounts of 1 to 5 wt. % can be particularly advantageous.

Cosmetic oils can furthermore additionally be used with the active ingredient combination (A) according to the invention. These oil bodies preferably have a melting point below 50° C., more preferably below 45° C., particularly preferably below 40° C., highly preferably below 35° C. and most preferably the cosmetic oils are free-flowing at a temperature below 30° C. These oils are defined and described in more detail below.

The natural and synthetic cosmetic oils include, for example:
vegetable oils. Examples of such oils are sunflower oil, olive oil, soybean oil, rapeseed oil, almond oil, jojoba oil, orange oil, wheat germ oil, peach kernel oil and the liquid components of coconut butter. Other triglyceride oils are also suitable, however, such as the liquid components of beef fat and synthetic triglyceride oils
liquid paraffin oils, isoparaffin oils and synthetic hydrocarbons and also di-n-alkyl ethers having in total between 12 and 36 C atoms, in particular between 12 and 24 C atoms, such as for example di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether and n-hexyl-n-undecyl ether and also di-tert-butyl ether, diisopentyl ether, di-3-ethyl decyl ether, tert-butyl-n-octyl ether, isopentyl-n-octyl ether and 2-methyl pentyl-n-octyl ether. The compounds 1,3-di-(2-ethylhexyl)cyclohexane and di-n-octyl ether, which are available as commercial products (Cetiol® S and Cetiol® OE respectively), can be preferred.

Suitable natural oils are for example amaranth seed oil, apricot kernel oil, argan oil, avocado oil, babassu oil, cottonseed oil, borage seed oil, camelina oil, thistle oil, groundnut oil, pomegranate kernel oil, grapefruit seed oil, hemp oil, hazelnut oil, elderberry seed oil, blackcurrant seed oil, jojoba oil, cocoa butter, linseed oil, macadamia nut oil, corn oil, almond oil, marula oil, evening primrose oil, olive oil, palm oil, rapeseed oil, rice oil, sea buckthorn fruit oil, sea buckthorn seed oil, sesame oil, shea butter, soybean oil, sunflower oil, grape seed oil, walnut oil or wild rose oil.

Solid paraffins or isoparaffins, carnauba waxes, beeswaxes, candelilla waxes, ozokerites, ceresin, spermaceti wax, sunflower wax, fruit waxes such as for example apple wax or citrus wax, PE or PP microwaxes can be used according to the invention as natural or synthetic waxes (Fatwax). Such waxes are available for example from Kahl & Co., Trittau.

The amount used is 0.1 to 50 wt. %, relative to the total agent, preferably 0.1 to 20 wt. % and more preferably 0.1 to 15 wt. %, relative to the total agent. The following cationic surfactants in accordance with formula (Tkat-2) can moreover be used.

RCO—X—N⁺R¹R²R³A⁻ (Tkat-2)

R here denotes a substituted or unsubstituted, branched or straight-chain alkyl or alkenyl residue having 11 to 35 carbon atoms in the chain,
X denotes —O—,
R¹ denotes an alkylene group having 2 to 6 C atoms, which can be non-substituted or substituted, wherein if it is substituted, substitution with an —OH or —NH group is preferred,
R², R³ independently of one another denote an alkyl or hydroxyalkyl group having 1 to 6 C atoms in the chain, wherein the chain can be straight or branched,
R⁵ denotes hydrogen or a C1 to C6 straight-chain or branched alkyl or alkenyl residue, which can also be substituted with a hydroxyl group.
Akypoquat® 131 is an example of such commercial products.

Esterquats according to the formula (Tkat1-2) can also be used.

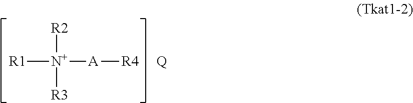

Here residues R1, R2 and R3 are independent of one another and can be identical or different. Residues R1, R2 and R3 denote:
a branched or unbranched alkyl residue having 1 to 4 carbon atoms, which can include at least one hydroxyl group, or
a saturated or unsaturated, branched or unbranched or a cyclic saturated or unsaturated alkyl residue having 6 to 30 carbon atoms, which can include at least one hydroxyl group, or
an aryl or alkaryl residue, for example phenyl or benzyl,
the residue (-A-R4), with the proviso that at most 2 of residues R1, R2 or R3 can denote this residue.

The residue -(A-R4) is included at least 1 to 3 times.

Here A denotes:
1) —(CH₂)n- where n=1 to 20, preferably n=1 to 10 and more preferably n=1 to 5, or
2) —(CH₂—CHR5-O)n- where n=1 to 200, preferably 1 to 100, more preferably 1 to 50,

and more preferably 1 to 20 with R5 denoting hydrogen, methyl or ethyl, and R4 denotes:
1) R6-O—CO—, in which R6 is a saturated or unsaturated, branched or unbranched or a cyclic saturated or unsaturated alkyl residue having 6 to 30 carbon atoms, which can include at least one hydroxyl group and which can optionally also be ethoxylated with 1 to 100 ethylene oxide units and/or 1 to 100 propylene oxide units, or
2) R7-CO—, in which R7 is a saturated or unsaturated, branched or unbranched or a cyclic saturated or unsaturated alkyl residue having 6 to 30 carbon atoms, which can include at least one hydroxyl group and which can optionally also be ethoxylated with 1 to 100 ethylene oxide units and/or 1 to 100 propylene oxide units,
   and Q denotes a physiologically acceptable organic or inorganic anion.

Such products are sold under the trademarks Rewoquat®, Stepantex®, Dehyquart® and Armocare®, for example. The products Armocare® VGH-70, an N,N-bis(2-palmitoyloxyethyl)dimethylammonium chloride, as well as Dehyquart® F-75, Dehyquart® C-4046, Dehyquart® L80, Dehyquart® F-30, Dehyquart® AU-35, Rewoquat® WE18, Rewoquat® WE38 DPG and Stepantex® VS 90 are examples of such esterquats.

Other compounds of formula (Tkat1-2) that are more preferred according to the invention are included in formula (Tkat1-2.1), cationic betaine esters.

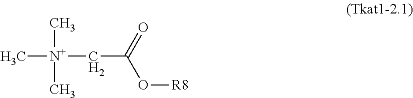

R8-, in which R8 is a saturated or unsaturated, branched or unbranched or a cyclic saturated or unsaturated alkyl residue having 6 to 30 carbon atoms, which can include at least one hydroxyl group and which can optionally also be ethoxylated with 1 to 100 ethylene oxide units and/or 1 to 100 propylene oxide units.

The anion of all cationic compounds is selected from the physiologically acceptable anions. The halide ions, fluoride, chloride, bromide; sulfate of the general formula RSO₃⁻, in which R has the meaning of saturated or unsaturated alkyl residues having 1 to 4 carbon atoms; or anionic residues of organic acids such as maleate, fumarate, propionate, oxalate, tartrate, citrate, lactate or acetate, are cited by way of example.

The aforementioned cationic surfactants can be used individually or in any combinations with one another, wherein they are included in amounts of between 0.01 and 20 wt. %, preferably in amounts from 0.01 to 10 wt. % and particularly preferably in amounts from 0.1 to 7.5 wt. %. The very best results are obtained with amounts from 0.1 to 5 wt. %, relative in each case to the total composition of the individual agent.

Emulsifiers that can be used according to the invention are for example
addition products of 4 to 30 mol of ethylene oxide and/or 0 to 5 mol of propylene oxide with linear fatty alcohols having 8 to 22 C atoms, with fatty acids having 12 to 22 C atoms and with alkyl phenols having 8 to 15 C atoms in the alkyl group,
C₁₂-C₂₂ fatty acid monoesters and diesters of addition products of 1 to 30 mol of ethylene oxide with polyols having 3 to 6 carbon atoms, in particular with glycerol,
ethylene oxide and polyglycerol addition products with methyl glucoside fatty acid esters, fatty acid alkanol amides,
addition products of 5 to 60 mol of ethylene oxide with castor oil and hydrogenated castor oil,
partial esters of polyols having 3 to 6 carbon atoms with saturated fatty acids having 8 to 22 C atoms,
sterols, from both animal tissue (zoosterols, cholesterol, lanosterol) and vegetable fats (phytosterols, ergosterol, stigmasterol, sitosterol) or from fungi and yeasts (mycosterols),
phospholipids (lecithins, phosphatidylcholines),
fatty acid esters of sugars and sugar alcohols, such as sorbitol,
polyglycerols and polyglycerol derivatives such as for example polyglycerol poly-12-hydroxystearate (commercial product Dehymuls® PGPH).

Further emulsifiers for the compositions according to the invention can be phospholipids. Both natural and synthetic phospholipids can be used as the phospholipid. Naturally based phospholipids are preferred. In particular, compounds of formula (Phosphol-I) can be used as phospholipids.

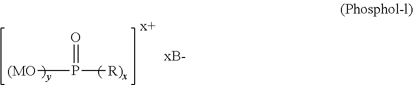

In formula (Phosphol-I) y denotes an integer from 0 to 2, x denotes an integer from 1 to 3 with the proviso that the sum of x and y is 3.

In the phospholipids of formula (Phosphol-I) M moreover denotes hydrogen, an equivalent of an alkali or alkaline-earth metal cation, an ammonium cation or an alkyl residue having 1 to 4 carbon atoms, which is optionally substituted with one or more hydroxyl groups. Compounds in which M denotes a sodium cation are more preferred.

Furthermore, B in formula (Phosphol-I) of the phospholipids for use according to the invention denotes an equivalent of a physiologically acceptable anion. Chloride, bromide, iodide, sulfate, perchlorate, tetrafluoroborate, tetraphenyl borate and tetrachlorozincate, for example, are suitable as the anion. The chloride ion is preferred.

R in the formula (Phosphol-I) denotes a residue of formula (II),

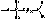

in which z denotes an integer from 1 to 4, in particular 3, and R¹ and R² independently of one another denote a C₁ to C₄ alkyl residue, which is optionally substituted with one or more hydroxyl groups or an acyl group.
According to the invention A denotes one of the units —O—CH₂—CH₂—CH₂—, —O—CH₂—CH₂— or —O—CH₂—CHOH—CH₂—, the unit —O—CH₂—CHOH—CH₂— being more preferred.
The residue R³ denotes
(a) a branched or unbranched, saturated C₈ to C₁₈ acyl residue or
(b) a branched or unbranched, mono- or polyunsaturated C₈ to C₁₈ acyl residue.

More preferred saturated residues R³ are the residue of stearic acid and the residues of the mixture of fatty acids obtained from coconut oil.

A more preferred unsaturated residue R³ is the residue of linoleic acid.

Examples of the C₁ to C₄ alkyl groups named as substituents in the compounds according to the invention are the methyl, ethyl, propyl, isopropyl and butyl groups. Ethyl and methyl groups are preferred alkyl groups. Methyl groups are particularly preferred.

Particularly preferred phospholipids of formula (Phosphol-I) are the substances known under the INCI names Linoleamidopropyl PG-Dimonium Chloride Phosphate, Cocamidopropyl PG-Dimonium Chloride Phosphate and Stearamidopropyl PG-Dimonium Chloride Phosphate. These are sold by Mona for example under the commercial names Phospholipid EFA®, Phospholipid PTC® and Phospholipid SV®.

Furthermore, the glycerophospholipids obtained for example as lecithins or phosphatidylcholines from egg yolk or plant seeds, in particular soybeans, for example, are used as phospholipids according to the invention. Phospholipids are in particular phosphoglycerides.

Glycerophospholipids that are particularly suitable according to the invention are obtained from soybeans. Of these, the phosphatidylcholines, phosphatidylethanolamines, phosphatidylserines and phosphatidylinositols and mixtures of these substances are more preferred.

The more preferred phosphatidylcholines have the formula (Phosphol-II),

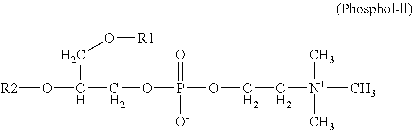

in which the residues R¹ and R² each independently of one another denote an acyl group of fatty acids with 8-30 C atoms, preferably 10-24 and more preferably 12-22 C atoms. The fatty acid residues can be both saturated and mono- or polyunsaturated. The saturated acyl residues of C₁₂-C₂₂ fatty acids are preferred. The acyl residues of myristic acid, palmitic acid, stearic acid, eicosanoic acid and docosanoic acid are more preferred.

The phosphatidylethanolamines that are preferred according to the invention are those of formula (Phosphol-IIa) or (Phosphol-IIb),

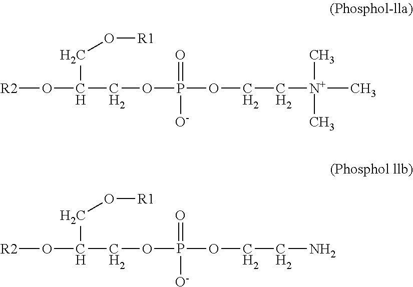

in which the residues R¹ and R² have the same meaning as for formula (Phosphol-III). Phosphatidylethanolamines in which R¹ and R² independently of one another denote saturated acyl residues of fatty acids with 16 or 18 carbon atoms, in particular a palmitoyl or stearoyl residue, are more preferred.

The phosphatidylserines that are preferred according to the invention are those of structural formula (IIIc) or (IIIb),

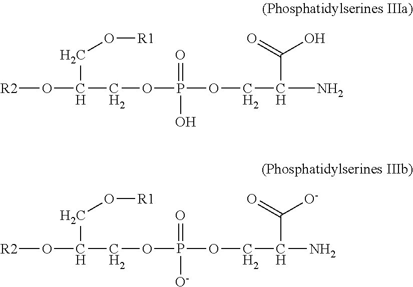

in which R¹ and R² have the same meaning as for formula (Phosphol-II). Phosphatidylserines in which R¹ and R² independently of one another denote saturated acyl residues of fatty acids with 16 or 18 carbon atoms, in particular a palmitoyl or stearoyl residue, are more preferred.
The phosphatidylinositols that are preferred according to the invention have structural formula (IVa) or (IVb),

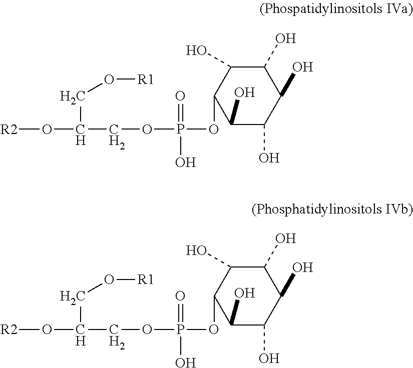

in which the residues R¹ and R² have the same meaning as for formula (Phosphol-II). For R¹ acyl residues of palmitic acid, stearic acid and eicosanoic acid are preferred; a stearic acid acyl residue is more preferred. R² more preferably denotes a linear saturated C₂₀ fatty acid acyl residue (arachoyl residue).

The glycerophospholipids used according to the invention have an iodine value of at most 10, preferably at most 5.

It is also possible according to the invention to use a mixture of several phospholipids. A phospholipid that is preferred according to the invention is commercially available under the name Emulmetik® 100 (Cognis). The phospholipids according to the invention are included in the agents in concentrations of 0.1 wt. % to 7.5 wt. %, preferably 0.1 wt. % to 5 wt. %, particularly preferably in amounts from 0.1 wt. % to 3 wt. % and highly preferably in amounts from 0.1 to 1.5 wt. %.

The agents according to the invention include emulsifiers preferably in amounts from 0.1 to 25 wt. %, in particular 0.5 to 15 wt. %, relative to the total agent.

The cationic polymers can be homo- or copolymers, wherein the quaternary nitrogen groups are included either in the polymer chain or preferably as a substituent at one or more of the monomers. The ammonium group-including monomers can be copolymerized with non-cationic monomers. Suitable cationic monomers are unsaturated, radically polymerizable compounds bearing at least one cationic group, in particular ammonium-substituted vinyl monomers such as for example trialkyl methacryloxy alkylammonium, trialkyl acryloxy alkylammonium, dialkyl diallyl ammonium and quaternary vinyl ammonium monomers with cyclic groups including cationic nitrogens, such as pyridinium, imidazolium or quaternary pyrrolidones, e.g. alkyl vinyl imidazolium, alkyl vinyl pyridinium, or alkyl vinyl pyrrolidone salts. The alkyl groups of these monomers are preferably low alkyl groups such as for example C1 to C7 alkyl groups, more preferably C1 to C3 alkyl groups.

The ammonium group-including monomers can be copolymerized with non-cationic monomers. Suitable comonomers are for example acrylamide, methacrylamide, alkyl and dialkyl acrylamide, alkyl and dialkyl methacrylamide, alkyl acrylate, alkyl methacrylate, vinyl caprolactone, vinyl caprolactam, vinyl pyrrolidone, vinyl esters, for example vinyl acetate, vinyl alcohol, propylene glycol or ethylene glycol, the alkyl groups of these monomers preferably being C1 to C7 alkyl groups, more preferably C1 to C3 alkyl groups.

Suitable polymers having quaternary amine groups are for example the polymers described in the CTFA Cosmetic Ingredient Dictionary under the name Polyquaternium, such as methylvinyl imidazolium chloride/vinyl pyrrolidone copolymer (Polyquaternium-16) or quaternized vinyl pyrrolidone/dimethyl aminoethyl methacrylate copolymer (Polyquaternium-11).

Of the cationic polymers that can be included in the agent according to the invention, vinyl pyrrolidone/dimethylaminoethyl methacrylate methosulfate copolymer for example, which is sold under the trade names Gafquat® 755 N and Gafquat® 734 by Gaf Co., USA, is suitable, of which Gafquat® 734 is more preferred. Further cationic polymers are for example the copolymer of polyvinyl pyrrolidone and imidazolimine methochloride, which is sold by BASF, Germany under the trade name Luviquat® HM 550, the terpolymer of dimethyldiallyl ammonium chloride, sodium acrylate and acrylamide, which is sold by Calgon/USA under the trade name Merquat® Plus 3300, and the vinyl pyrrolidone/methacrylamidopropyl trimethylammonium chloride copolymer sold by ISP under the trade name Gafquat® HS 100.

Homopolymers of the general formula (P1), —{CH₂-[CR¹COO—(CH₂)ₘN⁺R²R³R⁴]}ₙX⁻,

in which R¹=—H or —CH₃, R², R³ and R⁴ are selected independently of one another from C1-4 alkyl, alkenyl or hydroxyalkyl groups, m=1, 2, 3 or 4, n is a natural number and X⁻ is a physiologically acceptable organic or inorganic anion. In the context of these polymers, those for which at least one of the following conditions applies are preferred according to the invention: R¹ denotes a methyl group, R², R³ and R⁴ denote methyl groups, m has the value 2.

Suitable physiologically acceptable counterions X⁻ are for example halide ions, sulfate ions, phosphate ions, methosulfate ions as well as organic ions such as lactate, citrate, tartrate and acetate ions. Halide ions, in particular chloride, are preferred.

Suitable cationic guar derivatives are sold under the trade name Jaguar® and have the INCI name Guar Hydroxypropyltrimonium Chloride. Particularly suitable cationic guar derivatives are also sold furthermore by Hercules under the name N-Hance®. Further cationic guar derivatives are sold by Cognis under the name Cosmedia®. A preferred cationic guar derivative is the commercial product AquaCat® from Hercules. This raw material is a pre-dissolved cationic guar derivative.

A suitable chitosan is sold for example by Kyowa Oil & Fat, Japan, under the trade name Flonac®. A preferred chitosan salt is chitosonium pyrrolidone carboxylate, which is sold for example under the name Kytamer® PC by Amerchol, USA. Further chitosan derivatives are freely available commercially under the trade names Hydagen® CMF, Hydagen® HCMF and Chitolam® NB/101.

Further preferred cationic polymers are for example
cationic alkyl polyglycosides,
cationized honey, for example the commercial product Honeyquat® 50,
polymeric dimethyl diallyl ammonium salts and copolymers thereof with esters and amides of acrylic acid and methacrylic acid. The products available commercially under the names Merquat® 100 (poly(dimethyl diallyl ammonium chloride)) and Merquat® 550 (dimethyl diallyl ammonium chloride acrylamide copolymer) are examples of such cationic polymers,
vinyl pyrrolidone-vinyl imidazolium methochloride copolymers, such as are sold under the names Luviquat® FC 370, FC 550, FC 905 and HM 552,
quaternized polyvinyl alcohol,
and the polymers known under the names Polyquaternium-2, Polyquaternium-17, Polyquaternium-18 and Polyquaternium-27 with quaternary nitrogen atoms in the polymer main chain,
vinyl pyrrolidone-vinyl caprolactam-acrylate terpolymers, such as are available commercially with acrylic acid esters and acrylic acid amides as the third monomer unit under the name Aquaflex® SF 40, for example.

Likewise suitable for use according to the invention are the copolymers of vinyl pyrrolidone, such as are available as the commercial products Copolymer 845 (manufacturer: ISP), Gaffix® VC 713 (manufacturer: ISP), Gafquat® ASCP 1011, Gafquat® HS 110, Luviquat® 8155 and Luviquat® MS 370.

The cationic polymers also include cationized protein hydrolysates, wherein the underlying protein hydrolysate can derive from animal sources, for example from collagen, milk or keratin, from plant sources, for example from wheat, corn, rice, potatoes, soy or almonds, from marine life forms, for example from fish collagen or algae, or from protein hydrolysates obtained by biotechnology. Typical examples of the cationic protein hydrolysates and derivatives according to the invention are the products that are listed under the INCI names in the “International Cosmetic Ingredient Dictionary and Handbook”, (seventh edition 1997, The Cosmetic, Toiletry, and Fragrance Association, 1101 17th Street, N.W., Suite 300, Washington, D.C. 20036-4702) and that are available commercially.

The additional cationic polymers are included in the compositions according to the invention preferably in amounts from 0.01 to 10 wt. %, relative to the total agent. Amounts from 0.05 to 5 wt. % are more preferred.

Amphoteric polymers can also be used as polymers. The term amphoteric polymers includes both polymers which include both free amino groups and free —COOH or SO₃H groups in the molecule and which are capable of forming internal salts, and zwitterionic polymers which include quaternary ammonium groups and —COO⁻ or —SO₃⁻ groups in the molecule, and such polymers including —COOH or SO₃H groups and quaternary ammonium groups.

Amphoteric and/or cationic polymers that are preferred according to the invention are polymers in which a cationic group is derived from at least one of the following monomers:
(i) monomers having quaternary ammonium groups of the general formula (Mono1),

R¹—CH═CR²—CO—Z—(CₙH₂ₙ)—N⁽⁺⁾R²R³R⁴A⁽⁻⁾ (Mono1)
in which R¹ and R² independently of one another denote hydrogen or a methyl group and R³, R⁴ and R⁵ independently of one another denote alkyl groups having 1 to 4 carbon atoms, Z is an NH group or an oxygen atom, n is an integer from 2 to 5 and A⁽⁻⁾ is the anion of an organic or inorganic acid,
(ii) monomers having quaternary ammonium groups of the general formula (Mono2),

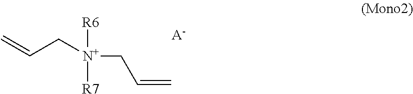

in which R⁶ and R⁷ independently of one another denote a (C₁ to C₄)alkyl group, in particular a methyl group, and
A⁻ is the anion of an organic or inorganic acid,
(iii) monomeric carboxylic acids of the general formula (Mono3),

R⁸—CH═CR⁹—COOH (Mono3)
in which R⁸ and R⁹ are independently of one another hydrogen or methyl groups.

Polymers in which monomers of type (i) are used, in which R³, R⁴ and R⁵ are methyl groups, Z is an NH group and A⁽⁻⁾ is a halide, methoxy sulfate or ethoxy sulfate ion, are more preferred; acrylamidopropyl trimethylammonium chloride is a more preferred monomer (i). Acrylic acid is preferably used as the monomer (ii) for the cited polymers.

More preferred amphoteric polymers are copolymers of at least one monomer (Mono1) or (Mono2) with the monomer (Mono3), in particular copolymers of monomers (Mono2) and (Mono3). Amphoteric polymers that are particularly preferably used according to the invention are copolymers of diallyl dimethylammonium chloride and acrylic acid. These copolymers are sold under the INCI name Polyquaternium-22, inter alia under the trade name Merquat® 280 (Nalco).

In addition to a monomer (Mono 1) or (Mono2) and a monomer (Mono3), the amphoteric polymers according to the invention can moreover additionally include a monomer (Mono4)
(iv) monomeric carboxylic acid amides of the general formula (Mono4),

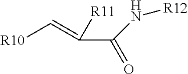

in which R¹⁰ and R¹¹ are independently of one another hydrogen or methyl groups and R¹² denotes a hydrogen atom or a (C₁ to C₈)alkyl group.

Amphoteric polymers based on a comonomer (Mono4) that are particularly preferably used according to the invention are terpolymers of diallyl dimethylammonium chloride, acrylamide and acrylic acid. These copolymers are sold under the INCI name Polyquaternium-39, inter alia under the trade name Merquat® Plus 3330 (Nalco).

The amphoteric polymers can generally be used according to the invention both directly and in the form of the salt, which is obtained by neutralization of the polymers, with an alkali hydroxide for example.

The amphoteric polymers are included in the agents according to the invention preferably in amounts from 0.01 to 10 wt. %, relative to the total agent. Amounts from 0.01 to 5 wt. % are more preferred.

The anionic polymers are anionic polymers having carboxylate and/or sulfonate groups. Examples of anionic monomers which can constitute such polymers are acrylic acid, methacrylic acid, crotonic acid, maleic anhydride and 2-acrylamido-2-methylpropane sulfonic acid. Some or all of the acid groups therein can be present as the sodium, potassium, ammonium, mono- or triethanolammonium salt. Preferred monomers are 2-acrylamido-2-methylpropane sulfonic acid and acrylic acid.

Anionic polymers including as the sole monomer or as a co-monomer 2-acrylamido-2-methylpropane sulfonic acid, in which some or all of the sulfonic acid group can be present as the sodium, potassium, ammonium, mono- or triethanolammonium salt, have proved to be particularly effective.

The homopolymer of 2-acrylamido-2-methylpropane sulfonic acid, which is available commercially for example under the name Rheothik®11-80, is more preferred.

Within this embodiment it can be preferable to use copolymers consisting of at least one anionic monomer and at least one non-ionogenic monomer. Reference is made to the aforementioned substances with regard to the anionic monomers. Preferred non-ionogenic monomers are acrylamide, methacrylamide, acrylic acid ester, methacrylic acid ester, vinyl pyrrolidone, vinyl ether and vinyl ester.

Preferred anionic copolymers are acrylic acid-acrylamide copolymers as well as in particular polyacrylamide copolymers with monomers including sulfonic acid groups. Such a polymer is included in the commercial product Sepigel® 305 from SEPPIC.

The sodium acryloyldimethyltaurate copolymers sold under the name Simulgel® 600 as a compound with isohexadecane and Polysorbate-80 have also proved to be particularly effective according to the invention.

Likewise preferred anionic homopolymers are uncrosslinked and crosslinked polyacrylic acids. Allyl ethers of pentaerythritol, of sucrose and of propylene can be preferred crosslinking agents here. Such compounds are available commercially for example under the trademark Carbopol®.

Copolymers of maleic anhydride and methyl vinyl ether, in particular those with crosslinkages, are likewise color-retaining polymers. A maleic acid-methyl vinyl ether copolymer crosslinked with 1,9-decadiene is available commercially under the name Stabileze® QM.

The anionic polymers are included in the agents according to the invention preferably in amounts from 0.05 to 10 wt. %, relative to the total agent. Amounts from 0.1 to 5 wt. % are more preferred.

A polyurethane that is particularly preferred according to the invention is sold under the trade name Luviset® PUR (BASF).

In a further embodiment the agents according to the invention can include non-ionogenic polymers.

Suitable non-ionogenic polymers are for example:
vinyl pyrrolidone/vinyl ester copolymers, such as are sold for example under the trademark Luviskol® (BASF). Luviskol® VA 64 and Luviskol® VA 73, both of which are vinyl pyrrolidone/vinyl acetate copolymers, are likewise preferred non-ionic polymers;
cellulose ethers, such as hydroxypropyl cellulose, hydroxyethyl cellulose and methylhydroxypropyl cellulose, such as are sold for example under the trademarks Culminal® and Benecel® (AQUALON), and Natrosol® types (Hercules),
starch and derivatives thereof, in particular starch ethers, for example Structure® XL (National Starch), a multifunctional, salt-tolerant starch;
shellac;
polyvinyl pyrrolidones, such as are sold for example under the name Luviskol® (BASF).

The non-ionic polymers are included in the compositions according to the invention preferably in amounts from 0.05 to 10 wt. %, relative to the total agent. Amounts from 0.1 to 5 wt. % are more preferred.

It is also possible according to the invention for the preparations used to include a plurality of, in particular two, different polymers of the same charge and/or an ionic and an amphoteric and/or non-ionic polymer.

The polymers (P) are included in the compositions used according to the invention preferably in amounts from 0.01 to 30 wt. %, relative to the total composition. Amounts from 0.01 to 25, in particular from 0.01 to 15 wt. %, are more preferred.

As a further optional ingredient the agents according to the invention preferably include at least one silicone polymer selected from the group of dimethiconols and/or the group of amino-functional silicones and/or the group of dimethicones and/or the group of cyclomethicones. These ingredients are described below.

The dimethicones according to the invention can be both linear and branched and also cyclic or cyclic and branched. Linear dimethicones can be represented by the following structural formula (Si1):

(SiR¹₃)—O—(SiR²₂—O—)ₓ—(SiR¹₃) (Si1)

Branched dimethicones can be represented by the structural formula (Si1.1):

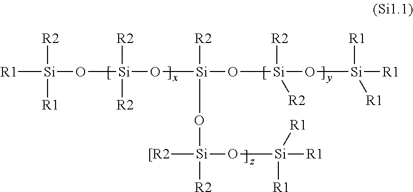

Residues R¹ and R² independently of one another denote hydrogen, a methyl residue, a C2 to C30 linear, saturated or unsaturated hydrocarbon residue, a phenyl residue and/or an aryl residue. The numbers x, y and z are integers and each run independently of one another from 0 to 50,000. The molecular weights of the dimethicones are between 1000 D and 10,000,000 D. The viscosities are between 100 and 10,000,000 cPs, measured at 25° C. with a glass capillary viscometer in accordance with the Dow Corning corporate test method CTM 0004 of 20 Jul. 1970. Preferred viscosities are between 1000 and 5,000,000 cPs, particularly preferred viscosities are between 10,000 and 3,000,000 cPs. The most preferred range is between 50,000 and 2,000,000 cPs. Viscosities around the range of approximately 60,000 cPs are highly preferred. Reference is made here by way of example to the product “Dow Corning 200 with 60,000 cSt”.

More preferred cosmetic or dermatological preparations according to the invention are characterized in that they include at least one silicone of formula (Si1.2)

(CH₃)₃Si—[O—Si(CH₃)₂]ₓ—O—Si(CH₃)₃ (Si1.2),

in which x denotes a number from 0 to 100, preferably from 0 to 50, more preferably from 0 to 20 and in particular 0 to 10.

The dimethicones (Si1) are included in the compositions according to the invention in amounts from 0.01 to 10 wt. %, preferably 0.01 to 8 wt. %, more preferably 0.1 to 7.5 wt. % and in particular 0.1 to 5 wt. %, relative to the total composition.

More preferred agents according to the invention include one or more amino-functional silicones. Such silicones can be described for example by the formula (Si-2)

M(RₐQ_{b}SiO_{(4-a-b)/2})ₓ(R_{c}SiO_{(4-c)/2})_{y}M (Si-2)

wherein in the above formula
R is a hydrocarbon or a hydrocarbon residue having 1 to approximately 6 carbon atoms,
Q is a polar residue of the general formula —R¹HZ,
   in which
   R¹ is a divalent linking group that is bonded to hydrogen and to the residue Z, composed of carbon and hydrogen atoms, carbon, hydrogen and oxygen atoms or carbon, hydrogen and nitrogen atoms, and
   Z is an organic, amino-functional residue including at least one amino-functional group;
a assumes values in the range from approximately 0 to approximately 2,
b assumes values in the range from approximately 1 to approximately 3,
a+b is less than or equal to 3, and
c is a number in the range from approximately 1 to approximately 3, and
x is a number in the range from 1 to approximately 2000, preferably from approximately 3 to approximately 50 and most preferably from approximately 3 to approximately 25, and
y is a number in the range from approximately 20 to approximately 10,000, preferably from approximately 125 to approximately 10,000 and most preferably from approximately 150 to approximately 1000, and
M is a suitable silicone end group as is known in the prior art, preferably trimethylsiloxy.

According to formula (Si-2) Z is an organic, amino-functional residue including at least one functional amino group. A possible formula for said Z is NH(CH₂)_{z}NH₂, in which z is an integer greater than or equal to 1. Another possible formula for said Z is —NH(CH₂)_{z}(CH₂)_{zz}NH, in which both z and zz are independently of one another an integer greater than or equal to 1, this structure comprising diamino ring structures, such as piperazinyl. Said Z is most preferably an —NHCH₂CH₂NH₂ residue. Another possible formula for said Z is —N(CH₂)_{z}(CH₂)_{zz}NX₂ or —NX₂, in which each X of X₂ is selected independently from the group consisting of hydrogen and alkyl groups having 1 to 12 carbon atoms, and zz is 0.

Q according to formula (Si-2) is most preferably a polar amino-functional residue of the formula —CH₂CH₂CH₂NHCH₂CH₂NH₂.

In formula (Si-2) a assumes values in the range from 0 to 2, b assumes values in the range from 2 to 3, a+b is less than or equal to 3, and c is a number in the range from 1 to 3. Cationic silicone oils such as for example the commercially available products Dow Corning (DC) 929 Emulsion, DC 2-2078, DC 5-7113, SM-2059 (General Electric) and SLM-55067 (Wacker) are suitable according to the invention.

More preferred agents according to the invention are characterized in that they include at least one amino-functional silicone of formula (Si-3a)

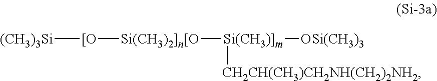

in which m and n are numbers whose sum (m+n) is between 1 and 2000, preferably between 50 and 150, wherein n preferably assumes values from 0 to 1999 and in particular from 49 to 149 and m preferably assumes values from 1 to 2000, in particular from 1 to 10.

Under the INCI declaration these silicones are known as trimethylsilylamodimethicones and they are available for example under the name Q2-7224 (manufacturer: Dow Corning; a stabilized trimethylsilylamodi-methicone).

Also more preferred are agents according to the invention that include an amino-functional silicone of formula (Si-3b)

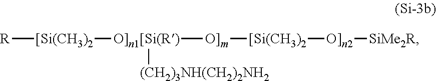

in which
R denotes —OH, an (optionally ethoxylated and/or propoxylated) (C₁ to C₂₀)alkoxy group or a —CH₃ group,
R′ denotes —OH, a (C₁ to C₂₀)alkoxy group or a —CH₃ group, and
m, n1 and n2 are numbers whose sum (m+n1+n2) is between 1 and 2000, preferably between 50 and 150, wherein the sum (n1+n2) preferably assumes values from 0 to 1999 and in particular from 49 to 149 and m preferably assumes values from 1 to 2000, in particular from 1 to 10.

Under the INCI declaration these silicones are known as amodimethicone or as functionalized amodimethicone, such as for example Bis(C13-15 Alkoxy) PG Amodimethicone (available for example as the commercial product DC 8500 from Dow Corning), Trideceth-9 PG-Amodimethicone (available for example as the commercial product Silcare Silicone SEA from Clariant).

Suitable diquaternary silicones are selected from compounds of the general formula (Si3c)

[R¹R²R³N⁺-A-SiR⁷R⁸—)(O—SiR⁹R¹⁰)ₙ—O—SiR¹¹R¹²-A-N⁺R⁴R⁵R⁶]2X⁻ (Si3c)

in which residues R1 to R6 independently of one another denote C1 to C22 alkyl residues, which can include hydroxyl groups, and wherein preferably at least one of the residues has at least 8 C atoms and the other residues have 1 to 4 C atoms,
residues R7 to R12 independently of one another are identical or different and denote C1 to C10 alkyl or phenyl, A denotes a divalent organic group of compounds,
n is a number from 0 to 200, preferably from 10 to 120, more preferably from 10 to 40, and X⁻ is an anion.

The divalent group of compounds is preferably a C1 to C12 alkylene or alkoxyalkylene group, which can be substituted with one or more hydroxyl groups. The group —(CH₂)₃—O—CH₂—CH(OH)—CH₂— is more preferred.

The anion X⁻ can be a halide ion, an acetate, an organic carboxylate or a compound of the general formula RSO₃⁻, in which R has the meaning of C1 to C4 alkyl residues.

A preferred diquaternary silicone has the general formula (Si3d)

[RN⁺Me₂-A-(SiMe₂O)ₙ—SiMe₂-A-N⁺Me₂R]2CH₃COO⁻ (Si3d),

in which A is the group —(CH₂)₃—O—CH₂—CH(OH)—CH₂—,
R is an alkyl residue having at least 8 C atoms and n is a number from 10 to 120.

Suitable silicone polymers having two terminal, quaternary ammonium groups are known under the INCI name Quaternium-80. These are dimethyl siloxanes having two terminal trialkylammonium groups. Such diquaternary polydimethyl siloxanes are sold by Evonik under the trade names Abil® Quat 3270, 3272 and 3474.

Cosmetic or dermatological preparations that are preferred according to the invention are characterized in that they include, relative to their weight, 0.01 to 10 wt. %, preferably 0.01 to 8 wt. %, more preferably 0.1 to 7.5 wt. % and in particular 0.2 to 5 wt. % of amino-functional silicone(s) and/or diquaternary silicone.

The compositions according to the invention can include at least one polyammonium-polysiloxane compound as the silicone. The polyammonium-polysiloxane compounds can be purchased for example from GE Bayer Silicones under the trade name Baysilone®. The products with the names Baysilone TP 3911, SME 253 and SFE 839 are preferred here. The use of Baysilone TP 3911 as the active component of the compositions according to the invention is particularly preferred. The polyammonium-polysiloxane compounds are used in the compositions according to the invention in an amount from 0.01 to 10 wt. %, preferably 0.01 to 7.5, more preferably 0.01 to 5.0 wt. %, particularly preferably from 0.05 to 2.5 wt. %, relative in each case to the total composition.

Further cationic amino-functional silicone polymers can moreover also be used. EP 1887024 A1 describes novel cationic amino-functional silicones, which in particular improve the shine in care agents for surfaces, for example human hair. These cationic silicone polymers are characterized in that they have a silicone framework and at least one polyether part and moreover at least one part having an ammonium structure. Examples of the preferred cationic silicone polymers within the meaning of the present invention, in addition to the compounds of the aforementioned EP 1887024 A1, are moreover in particular the compounds having the INCI names: Silicone Quaternium-1, Silicone Quaternium-2, Silicone Quaternium-3, Silicone Quaternium-4, Silicone Quaternium-5, Silicone Quaternium-6, Silicone Quaternium-7, Silicone Quaternium-8, Silicone Quaternium-9, Silicone Quaternium-10, Silicone Quaternium-11, Silicone Quaternium-12, Silicone Quaternium-15, Silicone Quaternium-16, Silicone Quaternium-17, Silicone Quaternium-18, Silicone Quaternium-20, Silicone Quaternium-21, Silicone Quaternium-22 and Silicone Quaternium-2 Panthenol Succinate and Silicone Quaternium-16/Glycidyl Dimethicone Crosspolymer. Silicone Quaternium-22 is most preferred in particular. This raw material is sold for example by Evonik under the trade name Abil® T Quat 60.

The aforementioned cationic amino-functional silicone polymers are included in the compositions according to the invention in amounts from 0.01 to 20 wt. %, preferably in amounts from 0.05 to 10 wt. % and particularly preferably in amounts from 0.1 to 7.5 wt. %. The very best results are obtained with amounts from 0.1 to 5 wt. %, relative in each case to the total composition of the individual agent. According to the invention it can be particularly advantageous to use exclusively the aforementioned silicones as the silicones.

The cyclic dimethicones referred to under INCI as cyclomethicones can also be used to advantage according to the invention. Cosmetic or dermatological preparations according to the invention are preferred that include at least one silicone of formula (Si-4),

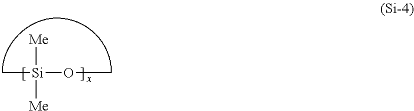

in which x denotes a number from 3 to 200, preferably from 3 to 10, more preferably from 3 to 7 and in particular 3, 4, 5 or 6.

Agents that are likewise preferred according to the invention are characterized in that they include at least one silicone of formula (Si-5)

R₃Si-[O—SiR₂]ₓ—(CH₂)ₙ[O—SiR₂]_{y}—O—SiR₃ (Si-5),

in which R denotes identical or different residues from the group —H, -phenyl, -benzyl, —CH₂—CH(CH₃)Ph, C₁-C₂₀ alkyl residues, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, x and y denote a number from 0 to 200, preferably from 0 to 10, more preferably from 0 to 7 and in particular 0, 1, 2, 3, 4, 5 or 6, and n denotes a number from 0 to 10, preferably from 1 to 8 and in particular 2, 3, 4, 5, 6.

As further silicones in addition to the dimethicones, dimethiconols, amodimethicones and/or cyclomethicones, water-soluble silicones can be included in the compositions according to the invention.

Corresponding hydrophilic silicones are selected for example from the compounds of formulae (Si-6) and/or (Si-7). Silicone-based water-soluble surfactants that are preferred in particular are selected from the group of dimethicone copolyols, which are preferably alkoxylated, in particular polyethoxylated or polypropoxylated.

According to the invention dimethicone copolyols are understood to be preferably polyoxyalkylene-modified dimethyl polysiloxanes of the general formulae (Si-6) or (Si-7):

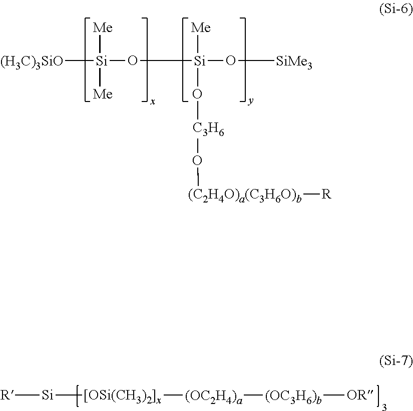

in which the residue R denotes a hydrogen atom, an alkyl group having 1 to 12 C atoms, an alkoxy group having 1 to 12 C atoms or a hydroxyl group, the residues R′ and R″ denote alkyl groups having 1 to 12 C atoms, x denotes an integer from 1 to 100, preferably from 20 to 30, y denotes an integer from 1 to 20, preferably from 2 to 10, and a and b denote integers from 0 to 50, preferably from 10 to 30.

More preferred dimethicone copolyols within the meaning of the invention are for example the products sold commercially under the trade name SILWET (Union Carbide Corporation) and DOW CORNING. Dimethicone copolyols that are more preferred according to the invention are Dow Corning 190 and Dow Corning 193.

The dimethicone copolyols are included in the compositions according to the invention in amounts from 0.01 to 10 wt. %, preferably 0.01 to 8 wt. %, more preferably 0.1 to 7.5 wt. % and in particular 0.1 to 5 wt. % of dimethicone copolyol, relative to the composition.

Finally, the silicone compounds are understood to include dimethiconols (Si8). The dimethiconols according to the invention can be both linear and branched and also cyclic or cyclic and branched. Linear dimethiconols can be represented by the following structural formula (Si8-I):

(SiOHR¹₂)—O—(SiR²₂—O—)ₓ—(SiOHR¹₂) (Si8-I)

Branched dimethiconols can be represented by the structural formula (Si8-II):

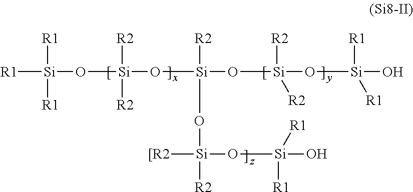

Residues R¹ and R² independently of one another denote hydrogen, a methyl residue, a C2 to C30 linear, saturated or unsaturated hydrocarbon residue, a phenyl residue and/or an aryl residue. The numbers x, y and z are integers and each run independently of one another from 0 to 50,000. The molecular weights of the dimethiconols are between 1000 D and 10,000,000 D. The viscosities are between 100 and 10,000,000 cPs, measured at 25° C. with a glass capillary viscometer in accordance with the Dow Corning corporate test method CTM 0004 of 20 Jul. 1970. Preferred viscosities are between 1000 and 5,000,000 cPs, particularly preferred viscosities are between 10,000 and 3,000,000 cPs. The most preferred range is between 50,000 and 2,000,000 cPs.

The following commercial products are cited as examples of such products: Dow Corning 1-1254 Fluid, Dow Corning 2-9023 Fluid, Dow Corning 2-9026 Fluid, Abil OSW 5 (Degussa Care Specialties), Dow Corning 1401 Fluid, Dow Corning 1403 Fluid, Dow Corning 1501 Fluid, Dow Corning 1784 HVF Emulsion, Dow Corning 9546 Silicone Elastomer Blend, SM555, SM2725, SM2765, SM2785 (these last four all GE Silicones), Wacker-Belsil CM 1000, Wacker-Belsil CM 3092, Wacker-Belsil CM 5040, Wacker-Belsil DM 3096, Wacker-Belsil DM 3112 VP, Wacker-Belsil DM 8005 VP, Wacker-Belsil DM 60081 VP (these last all Wacker-Chemie GmbH).

The dimethiconols (Si8) are included in the compositions according to the invention in amounts from 0.01 to 10 wt. %, preferably 0.01 to 8 wt. %, more preferably 0.1 to 7.5 wt. % and in particular 0.1 to 5 wt % of dimethiconol, relative to the composition.

Protein hydrolysates and/or derivatives thereof (P) are a further synergistic active ingredient according to the invention in the compositions according to the invention with the active ingredient complex according to the invention.

According to the invention protein hydrolysates of both plant and animal or marine or synthetic origin can be used.

Animal protein hydrolysates are for example elastin, collagen, keratin, silk and milk protein hydrolysates, which can also be present in the form of salts. Such products are sold for example under the trademarks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex) and Kerasol® (Croda).

Also preferred according to the invention are plant protein hydrolysates, such as for example soy, almond, pea, moringa, potato and wheat protein hydrolysates. Such products are available for example under the trademarks Gluadin® (Cognis), DiaMin® (Diamalt), Lexein® (Inolex), Hydrosoy® (Croda), Hydrolupin® (Croda), Hydrosesame® (Croda), Hydrotritium® (Croda), Crotein® (Croda) and Puricare® LS 9658 from Laboratoires Sérobiologiques.

Further protein hydrolysates that are preferred according to the invention are of marine origin. They include for example collagen hydrolysates of fish or algae and protein hydrolysates of mussels or pearl hydrolysates. Examples of pearl extracts according to the invention are the commercial products Pearl Protein Extract BG® or Crodarom® Pearl.

The protein hydrolysates (P) are included in the compositions in concentrations from 0.001 wt. % to 20 wt. %, preferably from 0.05 wt. % to 15 wt. % and particularly preferably in amounts from 0.05 wt. % to 5 wt. %.

The effect of the compositions according to the invention can be further increased by means of a 2-pyrrolidinone-5-carboxylic acid and derivatives (J) thereof. The sodium, potassium, calcium, magnesium or ammonium salts are preferred, in which the ammonium ion bears one to three C₁ to C₄ alkyl groups in addition to hydrogen. The sodium salt is particularly preferred. The amounts used in the agents according to the invention are 0.05 to 10 wt. %, relative to the total agent, more preferably 0.1 to 5, and in particular 0.1 to 3 wt. %.

A further preferred group of ingredients of the compositions according to the invention with the active ingredient complex according to the invention are vitamins, provitamins or vitamin precursors.

Vitamins, provitamins and vitamin precursors are more preferred that are assigned to groups A, B, C, E, F and H.

The group of substances classed as vitamin A includes retinol (vitamin A₁) and 3,4-didehydroretinol (vitamin A₂). β-Carotene is the retinol provitamin. Suitable vitamin A components according to the invention are for example vitamin A acid and esters thereof, vitamin A aldehyde and vitamin A alcohol and esters thereof such as the palmitate and acetate. The agents according to the invention include the vitamin A component preferably in amounts from 0.05 to 1 wt. %, relative to the total preparation.

The vitamin B group or vitamin B complex includes inter alia:

Vitamin B₁ (thiamine)
Vitamin B₂ (riboflavin)
Vitamin B₃. The compounds nicotinic acid and nicotinic acid amide (niacinamide) are often included under this term. Preferred according to the invention is nicotinic acid amide, which is preferably included in the agents used according to the invention in amounts from 0.05 to 1 wt. %, relative to the total agent.
Vitamin B₅ (pantothenic acid, panthenol and pantolactone). Within the context of this group panthenol and/or pantolactone is preferably used. Derivatives of panthenol which can be used according to the invention are in particular the esters and ethers of panthenol as well as cationically derivatized panthenols. Individual representatives are for example panthenol triacetate, panthenol monoethyl ether and the monoacetate thereof, and cationic panthenol derivatives. Pantothenic acid is preferably used in the present invention as a derivative in the form of the more stable calcium salts and sodium salts (Ca pantothenate, Na pantothenate)
Vitamin B₆ (pyridoxine as well as pyridoxamine and pyridoxal).

The cited compounds of the vitamin B type, in particular vitamin B₃, B₅ and B₆, are included in the agents according to the invention preferably in amounts from 0.05 to 10 wt. %, relative to the total agent. Amounts from 0.1 to 5 wt. % are more preferred.

Vitamin C (ascorbic acid). Vitamin C is used in the agents according to the invention preferably in amounts from 0.1 to 3 wt. %, relative to the total agent. Use in the form of the palmitic acid ester, glucosides or phosphates can be preferred. Use in combination with tocopherols can likewise be preferred.

Vitamin E (tocopherols, in particular α-tocopherol). Tocopherol and derivatives thereof, which include in particular esters such as acetate, nicotinate, phosphate and succinate, are preferably included in the agents according to the invention in amounts from 0.05 to 1 wt. %, relative to the total agent.

Vitamin F. The term “vitamin F” is conventionally understood to mean essential fatty acids, in particular linoleic acid, linolenic acid and arachidonic acid.

Vitamin H. Vitamin H is the name given to the compound (3aS,4S,6aR)-2-oxohexahydrothienol[3,4-d]-imidazole-4-valeric acid, although this is now more widely known by the trivial name biotin. Biotin is preferably included in the agents according to the invention in amounts from 0.0001 to 1.0 wt. %, in particular in amounts from 0.001 to 0.01 wt. %.

The compositions according to the invention preferably include vitamins, provitamins and vitamin precursors from groups A, B, E and H. Panthenol, pantolactone, pyridoxine and derivatives thereof as well as nicotinic acid amide and biotin are more preferred.

A more preferred group of ingredients in the cosmetic compositions according to the invention is constituted by the betaines listed below: carnitine, carnitine tartrate, carnitine magnesium citrate, acetyl carnitine, betalains, 1,1-dimethyl proline, choline, choline chloride, choline bitartrate, choline dihydrogen citrate and the compound N,N,N-trimethylglycine, which is classed in the literature as betaine.

Carnitine, histidine, choline and betaine are preferably used. In a more preferred embodiment of the invention L-carnitine tartrate is used as the active ingredient.

A particularly essential ingredient is taurine and/or a taurine derivative. Taurine is understood to be exclusively 2-aminoethane sulfonic acid, and a derivative to be the explicitly cited taurine derivatives. The taurine derivatives are understood to be N-monomethyl taurine, N,N-dimethyltaurine, taurine lysylate, taurine tartrate, taurine omithate, lysyl taurine and ornithyl taurine. Further taurine derivatives within the meaning of the present invention are taurocholic acid and hypotaurine.

Agents according to the invention which, relative to their weight, include 0.0001 to 10.0 wt. %, preferably 0.0005 to 5.0 wt. %, more preferably 0.001 to 2.0 wt. % and in particular 0.001 to 1.0 wt. % of taurine and/or a taurine derivative are more preferred.

In a further embodiment that is preferred according to the invention the compositions according to the invention include bioquinones. In the agents according to the invention suitable bioquinones are understood to be one or more ubiquinones and/or plastoquinones. The ubiquinones that are preferred according to the invention have the following formula:

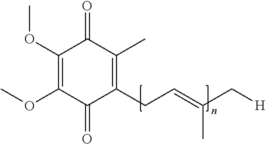

with n=6, 7, 8, 9 or 10.

Coenzyme Q-10 is most preferred here.

Preferred compositions according to the invention include purine and/or purine derivatives in relatively narrow quantity ranges. Cosmetic agents that are preferred according to the invention are characterized in that they include, relative to their weight, 0.001 to 2.5 wt. %, preferably 0.0025 to 1 wt. %, more preferably 0.005 to 0.5 wt. % and in particular 0.01 to 0.1 wt. % of purine(s) and/or purine derivative(s). Cosmetic agents that are preferred according to the invention are characterized in that they include purine, adenine, guanine, uric acid, hypoxanthine, 6-purinethiol, 6-thioguanine, xanthine, caffeine, theobromine or theophylline. In hair cosmetics preparations caffeine is most preferred.

In a further preferred embodiment of the present invention the cosmetic agent includes ectoine ((S)-2-methyl-1,4,5,6-tetrahydro-4-pyrimidine carboxylic acid).

More preferred according to the invention are agents which, relative to their weight, include 0.00001 to 10.0 wt. %, preferably 0.0001 to 5.0 wt. % and in particular 0.001 to 3 wt. % of active ingredients from the group formed from carnitine, taurine, coenzyme Q-10, ectoine, a vitamin of the B series, a purine and derivatives or physiologically acceptable salts thereof.

In a further embodiment the agents according to the invention should additionally include at least one UV light screening filter. UVB filters can be oil-soluble or water-soluble.

Examples of oil-soluble substances that can be cited include:
3-benzylidene camphor, for example 3-(4-methylbenzylidene)camphor;
4-aminobenzoic acid derivatives, preferably 4-(dimethylamino)benzoic acid-2-ethylhexyl ester, 4-(dimethylamino)benzoic acid-2-octyl ester and 4-(dimethylamino)benzoic acid amyl ester;
esters of cinnamic acid, preferably 4-methoxycinnamic acid-2-ethylhexyl ester, 4-methoxycinnamic acid propyl ester, 4-methoxycinnamic acid isoamyl ester, 2-cyano-3-phenylcinnamic acid-2-ethylhexyl ester (octocrylene);
esters of salicylic acid, preferably salicylic acid-2-ethylhexyl ester, salicylic acid-4-isopropylbenzyl ester, salicylic acid homomenthyl ester;
derivatives of benzophenone, preferably 2-hydroxy-4-methoxybenzophenone, 2-hydroxy-4-methoxy-4′-methylbenzophenone, 2,2′-dihydroxy-4-methoxybenzophenone;
esters of benzalmalonic acid, preferably 4-methoxybenzomalonic acid di-2-ethylhexyl ester;
triazine derivatives, such as for example 2,4,6-trianilino-(p-carbo-2′-ethyl-1′-hexyloxy)-1,3,5-triazine and octyl triazone;
propane-1,3-diones, such as for example 1-(4-tert-butylphenyl)-3-(4′-methoxyphenyl)propane-1,3-dione.

Suitable water-soluble substances include:
2-phenylbenzimidazole-5-sulfonic acid and alkali, alkaline-earth, ammonium, alkyl ammonium, alkanol ammonium and glucammonium salts thereof;
sulfonic acid derivatives of benzophenones, preferably 2-hydroxy-4-methoxybenzophenone-5-sulfonic acid and salts thereof;
sulfonic acid derivatives of 3-benzylidene camphor, such as for example 4-(2-oxo-3-bornylidene methyl)benzenesulfonic acid and 2-methyl-5-(2-oxo-3-bornylidene)sulfonic acid and salts thereof.

Derivatives of benzoyl methane are suitable in particular as typical UV-A filters, such as for example 1-(4′-tert-butylphenyl)-3-(4′-methoxyphenyl)propane-1,3-dione or 1-phenyl-3-(4′-isopropylphenyl) propane-1,3-dione. The UV-A and UV-B filters can naturally also be used in mixtures. In addition to the cited soluble substances, insoluble pigments, in particular finely dispersed metal oxides or salts, are suitable for this purpose, such as for example titanium dioxide, zinc oxide, iron oxide, aluminum oxide, cerium oxide, zirconium oxide, silicates (talc), barium sulfate and zinc stearate. The particles should have an average diameter of less than 100 nm, preferably between 5 and 50 nm and in particular between 15 and 30 nm. They can have a spherical form, but such particles having an ellipsoid form or other form deviating from the spherical shape can also be used.

Finally, further advantages arise through the use of plant extracts (L) in the compositions according to the invention. Preferred above all according to the invention are the extracts from green tea, oak bark, stinging nettle, witch hazel, hops, henna, chamomile, burdock, horsetail, whitethorn, lime blossom, almond, aloe vera, pine, horse chestnut, sandalwood, juniper, coconut, mango, apricot, lemon, wheat, kiwi, melon, orange, grapefruit, sage, rosemary, birch, mallow, valerian, lady's smock, wild thyme, yarrow, thyme, melissa, restharrow, coltsfoot, marshmallow, meristem, ginseng, coffee, cocoa, moringa, ginger root and ayurvedic plant extracts such as for example Aegle marmelos (bilva), Cyperus rotundus (nagarmotha), Emblica officinalis (amalaki), Morinda citrifolia (ashyuka), Tinospora cordifolia (guduchi), Santalum album (chandana), Crocus sativus (kumkuma), Cinnamonum zeylanicum and Nelumbo nucifera (kamala), grasses such as wheat, barley, rye, oats, spelt, corn, the various types of millet (proso millet, finger millet, foxtail millet as examples), sugar cane, ryegrass, meadow foxtail, false oat-grass, bentgrass, meadow fescue, moor grass, bamboo, cottongrass, pennisetums, Andropogonodeae (Imperata cylindrical, also known as blood grass or cogon grass), buffalo grass, cord grass, dog's tooth grass, lovegrass, Cymbopogon (citronella grass), Oryzeae (rice), Zizania (wild rice), marram grass, blue oatgrass, soft-grass, quaking grasses, speargrass, couch grass and Echinacea, in particular Echinacea angustifolia DC, Echinacea paradoxa (Norton), Echinacea simulata, E. atrorubens, E. tennesiensis, Echinacea strigosa (McGregor), Echinacea laevigata, Echinacea purpurea (L.) Moench and Echinacea pallida (Nutt), all types of vine and pericarp of Litchi chinensis.

The plant extracts can be used according to the invention in both pure and diluted form. If they are used in diluted form they conventionally include approximately 2 to 80 wt. % of active substance and as the solvent the extracting agent or mixture of extracting agents used to obtain them.

The cosmetic agents can furthermore include additional active ingredients, auxiliary substances and additives, such as for example
texturizing agents such as maleic acid and lactic acid,
swelling agents such as urea, allantoin, carbonates or hydantoin,
dimethyl isosorbide and cyclodextrins,
dyes to color the agent,
anti-dandruff active agents such as piroctone olamine, zinc omadine and climbazole,
complexing agents such as EDTA, NTA, β-alanine diacetic acid and phosphonic acids,
opacifiers such as latex, styrene/PVP and styrene/acrylamide copolymers,
pearlescent agents such as ethylene glycol mono- and distearate as well as PEG-3 distearate,
pigments,
stabilizing agents for hydrogen peroxide and other oxidizing agents,
propellants such as propane-butane mixtures, N₂O, dimethyl ether, CO₂ and air,
antioxidants, perfume oils, scents and fragrances.

With regard to further optional components and to the amounts of these components used, reference is expressly made to the relevant manuals known to the person skilled in the art.

As has already been mentioned, particular importance is attached to the good caring effect of the agents according to the invention. In addition to the highly preferred components of the active ingredient complex, in particular behentrimonium chloride, stearamidopropyl dimethylamine, isopropyl myristate and dicaprylyl carbonate, Polyquaternium-37 and lauryl glucoside, preferred agents according to the invention also include in a cosmetic carrier a cationic amino-functional silicone, highly preferably Silicone Quaternium-22, a vitamin of the B series and additionally at least one of the following active ingredients (i) to (viii):
(i) at least taurine or at least one taurine derivative,
(ii) at least one purine, in particular caffeine,
(iii) at least carnitine,
(iv) at least ectoine,
(v) at least one ubiquinone, in particular coenzyme Q-10,
(vi) at least one UV absorber,
(vii) at least one plant extract selected in particular from the extracts of hops, ginseng, Litchi chinensis or Echinacea,
(viii) ectoine.

The invention also provides the use of the composition according to the invention to improve the resistance of the surface of keratinic fibers to physical damage. Physical damage is understood according to the invention to mean the effect of UV light, the effect of heat from blow-drying, mechanical effects from combing and brushing the hair.

The present invention also provides the use of the composition according to the invention to improve the wash-out resistance of dyed keratinic fibers.

The invention also provides a method for hair treatment, in which a cosmetic agent according to claim 1 is applied to the hair and is rinsed out after a contact period.

The contact period is preferably a few seconds to 100 minutes, more preferably 1 to 50 minutes and particularly preferably 1 to 30 minutes.

A method in which a cosmetic agent according to claim 1 is applied to the hair and remains there is also in accordance with the invention. “Remains on the hair” is understood to mean according to the invention that the agent is not rinsed out of the hair again immediately after being applied. In this case the agent instead remains on the hair for more than 100 minutes and up to the next time the hair is washed.

### Examples

Unless otherwise specified, all stated amounts are parts by weight. The following formulations were prepared using known production methods.

| | | According to the |
|---|---|---|
| Ingredient | Comparison 1 | invention 1 (E1) |
| Cetearyl alcohol | 5.0 | 2.7 |
| Distearoylethyl | 1.0 | — |
| hydroxyethylmonium methosulfate | | |
| Ceteareth-20 | 0.3 | — |
| Cetrimonium chloride | 4.0 | — |
| Amodimethicone | 1.0 | — |
| Behentrimonium chloride | — | 1.8 |
| Stearamidopropyl dimethylamine | — | 0.4 |
| Glyceryl monostearate | 0.3 | 0.3 |
| Liquid paraffin | 0.8 | — |
| Isopropyl myristate | — | 0.8 |
| Citric acid | — | 0.5 |
| Lactic acid | 0.45 | — |
| Methylparaben Na salt | 0.3 | 0.3 |
| Polyquaternium-37 | — | 0.2 |
| Dicaprylyl carbonate | — | 0.15 |
| Lauryl glucoside | — | 0.01 |
| Panthenol | 0.2 | 0.2 |
| Nicotinic acid amide | 0.15 | 0.15 |
| Phenoxyethanol | 0.4 | 0.4 |
| Perfume | 0.3 | 0.3 |
| Water | to 100 | to 100 |
| pH | 2.5 | 2.5 |

At an independent testing institute for the cosmetics industry, the two formulations were tested on 20 women aged between 28 and 67 in a side-by-side test. The hair qualities hair length, hair thickness, greasiness and prior damage were measured. That means that all qualities were uniformly represented. Following the evaluation the parameters washability, distributability, skin feel, skin feel after rinsing, wet hair feel, detangling, wet hair combability, wet hair texture, curl retention, hair volume, antistatic properties and handling ability were significantly improved. All of the improved properties that were found are statistically significant.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic composition for conditioning keratinic fibers, including in a cosmetic carrier
a) 0.1 to 6.0 wt. % of cationic surfactant selected from behentrimonium chloride, dimethyl dibehenyl ammonium chloride, tribehenyl methyl trimonium chloride, cetyl trimethylammonium chloride, dicetyl dimethylammonium chloride, tricetyl methylammonium chloride, trimethyl stearyl ammonium chloride, dimethyl distearyl ammonium chloride, tristearyl methylammonium chloride, and mixtures thereof,
b) 0.01 to 6.0 wt. % of at least one amidoamine and/or one permanently cationic amidoamine,
c) 0.01 to 7.5 wt. % of at least one ester oil,
d) 0.01 to 5.0 wt. % of cationic polymer selected from Polyquaternium-67, Polyquaternium-72, Polyquaternium-74, and Polyquaternium-37, and mixtures thereof, and
e) 0.001 to 3.0 wt. % of at least one sugar surfactant, all stated amounts relating to the total weight of the cosmetic composition.

2. The cosmetic composition according to claim 1, wherein the ester oil is selected from the group consisting of symmetrical, asymmetrical or cyclic esters of carbonic acid with fatty alcohols.

3. The cosmetic composition according to claim 1, wherein the sugar surfactant is a C8 to C18 alkyl oligoglucoside.

4. The cosmetic composition according to claim 1, wherein the cationic polymer is Polyquaternium-37.

5. The cosmetic composition according to claim 1, wherein the cationic surfactant is selected from the group consisting of behentrimonium chloride and cetyltrimonium chloride.

6. The cosmetic composition according to claim 1, wherein the composition includes at least one silicone compound selected from the group consisting of Silicone Quaternium-1, Silicone Quaternium-2, Silicone Quaternium-3, Silicone Quaternium-4, Silicone Quaternium-5, Silicone Quaternium-6, Silicone Quaternium-7, Silicone Quaternium-8, Silicone Quaternium-9, Silicone Quaternium-10, Silicone Quaternium-11, Silicone Quaternium-12, Silicone Quaternium-15, Silicone Quaternium-16, Silicone Quaternium-17, Silicone Quaternium-18, Silicone Quaternium-20, Silicone Quaternium-21, Silicone Quaternium-22 and Silicone Quaternium-2 Panthenol Succinate and Silicone Quaternium-16/Glycidyl Dimethicone Crosspolymer.

7. The cosmetic composition according to claim 1, further comprising a vitamin of the B series.

8. A method for improving the resistance of the surface of keratinic fibers, comprising:
applying a composition according to claim 1 to washed and towel-dried keratinic fibers and leaving the composition there for a period from a few seconds to up to 10 minutes, and
rinsing the composition out of the keratinic fibers.
